SEQUENCE LISTING (4 sequences)

SEQ ID NO: 1 - protein, 8 aa, Artificial Sequence
  Linker molecule
FKGGERCG

SEQ ID NO: 2 - protein, 14 aa, Artificial Sequence
  Linker molecule
NQEQVSPLGGERCG

SEQ ID NO: 3 - protein, 10 aa, Artificial Sequence
  Cell adhesive peptide
RGDSPGERCG

SEQ ID NO: 4 - protein, 16 aa, Artificial Sequence
  matrix metalloprotease degradable crosslinker
GCRDGPQGIWGQDRCG

(19) United States
(12) Patent Application Publication
(10) Pub. No.: US 2020/0305773 A1
(43) Pub. Date: Oct. 1, 2020

(54) DEVICE AND METHOD FOR ANALYTE SENSING WITH MICROPOROUS ANNEALED PARTICLE GELS

(71) Applicant: THE REGENTS OF THE UNIVERSITY OF CALIFORNIA, Oakland, CA (US)

(72) Inventors: Dino Di Carlo, Los Angeles, CA (US); Westbrook Weaver, San Diego, CA (US); Donald Griffin, Charlottesville, VA (US); Jaekyung Koh, Los Angeles, CA (US)

(73) Assignee: THE REGENTS OF THE UNIVERSITY OF CALIFORNIA, Oakland, CA (US)

(21) Appl. No.: 16/071,252

(22) PCT Filed: Jan. 20, 2017
(86) PCT No.: PCT/US2017/014390

§ 371 Date: Jul. 19, 2018

Related U.S. Application Data
(60) Provisional application No. 62/281,660, filed on Jan. 21, 2016.

(51) Int. Cl. A61B 5/145; A61B 5/1473; A61B 5/00; A61B 5/1459; A61B 5/1455

(52) U.S. Cl. CPC A61B 5/14532; A61B 5/14735; A61B 5/0004; A61B 2562/02; A61B 5/742; A61B 5/1459; A61B 5/14556; A61B 5/4839

(57) ABSTRACT

A biocompatible analyte sensing material for intradermal or subcutaneous application includes a collection of microgel particles having one or more network crosslinker components and an endogenous or exogenous annealing agent that links the microgel particles together in situ to form a covalently-stabilized scaffold of microgel particles having interstitial spaces therein. A plurality of analyte-specific fluorophores are conjugated to the microgel particles, wherein the analyte-specific fluorophores, in the presence of the analyte and subject to excitation radiation, emit fluorescent light. The analyte sensing material may, in some embodiments, be applied to an excision made in the skin or injected into the skin of a subject.

### RELATED APPLICATION

This Application claims priority to U.S. Provisional Patent Application No. 62/281,660 filed on Jan. 21, 2016, which is hereby incorporated by reference in its entirety. Priority is claimed pursuant to 35 U.S.C. § 119 and any other applicable statute.

### TECHNICAL FIELD

The technical field generally relates to the field of biocompatible sensing materials that can measure and monitor analytes, drugs, or drug metabolites using microgel particles and scaffolds formed using the microgel particles.

### BACKGROUND

Materials that can seamlessly integrate with surrounding tissue at the microscale and are easily injected subcutaneously or to fill a wound, without a foreign body response, can be broadly useful in acute hemostasis, long-term regeneration of functional tissue, development of continuous implanted sensors, and sustained drug delivery. One new type of scaffold material has been developed that can accelerate wound healing. For example, Griffin et al. describe microporous annealed particle (MAP) gels that are delivered to a wound to form a MAP scaffold to accelerate wound healing. See Griffin et al., Accelerated wound healing by injectable microporous gel scaffolds assembled from annealed building blocks, Nature Materials, 14, 737-744 (2015).

This tissue scaffold material begins as an aqueous slurry of microfluidically fabricated microgel (μgel) building blocks that can be delivered to the desired site with a syringe applicator. Once applied to a wound, or injected subcutaneously, these spherical building blocks (˜100 microns in diameter) are triggered to anneal to surrounding μgel surfaces and the surrounding tissue, using clotting cascade enzymes or about 30 seconds of white light, to form an imperfect lattice-like structure with pores consisting of the interconnected void spaces between packed spherical particles. The engineered porosity of the scaffold accelerates infiltration of blood vessels and decreases fibrosis within the scaffold, instead supporting a natural-looking tissue. Microscale porosity also acts to prevent a foreign body response to the scaffold, which further reduces the fibrotic programs in the wound or encapsulation of the material. For example, once serum proteins decorate foreign materials, macrophages that are frustrated in a process of phagocytosis can assemble to form multinucleated giant cells that wall off and attempt to digest the implanted material. Over time, if this is unsuccessful, the sustained inflammatory response leads to formation of a fibrous capsule around the implant. This encapsulation by giant cells and a fibrotic capsule, however, reduces transport to the material from the surrounding circulation and tissue, and represents a key challenge that hinders regenerative healing, the long-term sensing of blood analytes, or effective delivery of drugs or analgesics.

### SUMMARY

In one embodiment, a biocompatible analyte sensing material for intradermal or subcutaneous application includes a collection of microgel particles comprising one or more network crosslinker components, wherein the microgel particles are spherical in shape and have diameters within the range from about 30 micrometers to about 150 micrometers. The material includes an endogenous or exogenous annealing agent that links the microgel particles together at points of physical contact in situ to form a covalently-stabilized scaffold of microgel particles having interstitial spaces therein. A plurality of analyte-specific fluorophores are conjugated to the microgel particles, wherein the analyte-specific fluorophores, in the presence of the analyte and subject to excitation radiation, emit fluorescent light.

In another embodiment, a system for sensing analyte concentrations in live tissue of a subject includes an analyte sensing material for intradermal or subcutaneous application comprising biocompatible microgel particles conjugated to analyte-specific fluorophores, wherein the analyte-specific fluorophores, in the presence of the analyte and when subject to excitation radiation, emit fluorescent light; and an optical readout device configured to illuminate the analyte sensing material with excitation radiation and read the intensity of emitted fluorescent light.

In still another embodiment, a method of sensing an analyte in a subject includes the operations of applying an analyte sensing material into an excision formed in the skin of a subject, the analyte sensing material comprising biocompatible microgel particles annealed or annealable to one another and conjugated to analyte-specific fluorophores, wherein the analyte-specific fluorophores, in the presence of the analyte and when subject to excitation radiation, emit fluorescent light; and exciting the analyte sensing material with excitation radiation and reading the emitted fluorescent light with an optical readout device.

In another embodiment, a method of sensing an analyte in a subject includes injecting an analyte sensing material into or under the skin of the subject, the analyte sensing material comprising biocompatible microgel particles annealed or annealable to one another and conjugated to analyte-specific fluorophores, wherein the analyte-specific fluorophores, in the presence of the analyte and when subject to excitation radiation, emit fluorescent light; and exciting the analyte sensing material with excitation radiation and reading the emitted fluorescent light with an optical readout device.

### BRIEF DESCRIPTION OF THE DRAWINGS

FIG. 1A illustrates a portion of region of a scaffold formed from a plurality of annealed microgel particles. The microgel particles have located thereon or therein analyte-specific fluorophores conjugated or bound to the microgel particles.

FIG. 1B illustrates a three dimensional analyte sensing scaffold that is formed intradermally within the skin tissue of a subject or patient.

FIG. 2A illustrates the sequence of operations used form three dimensional analyte sensing scaffold to an excision site.

FIG. 2B illustrates the sequence of operations used to inject the analyte sensing scaffold into a tissue site.

FIG. 2C illustrates the sequence of operations used to measure the concentration of an analyte using the analyte sensing scaffold. Also illustrated is an optional operation of using the measured concentration in combination with feedback control of a drug delivery device.

FIG. 3A illustrates one embodiment of an optical readout device that is used to deliver excitation radiation and receive emitted fluorescent light.

FIG. 3B illustrates one embodiment of an optical readout device that is positioned over an excision site containing the analyte sensing scaffold.

FIG. 3C illustrates another embodiment of an optical readout device.

FIG. 3D illustrates another embodiment of an optical readout device.

FIG. 4 illustrates a cross-sectional view of an optical readout device according to one embodiment that is positioned over the site containing the analyte sensing scaffold.

FIG. 5 illustrates an embodiment of an optical readout device that overlies a site containing the analyte sensing scaffold in form of a pattern.

FIG. 6 illustrates an embodiment of a pattern used to read different concentrations of an analyte.

FIG. 7 illustrates an embodiment in which the optical readout device communicates with a remote display device.

FIG. 8 illustrates another embodiment of a system in which the optical readout device communicates directly or indirectly to a drug delivery device such as a pump that is used to deliver a drug to the subject.

### DETAILED DESCRIPTION OF THE ILLUSTRATED EMBODIMENTS

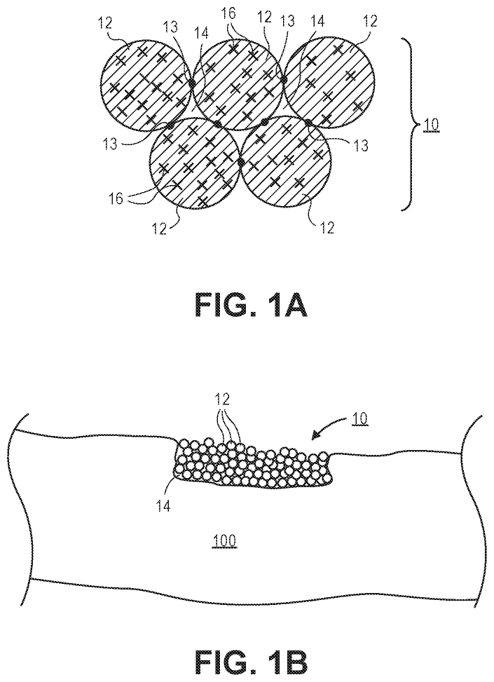

FIG. 1A illustrates a portion of the formed three dimensional scaffold 10 that is formed by a plurality of annealed microgel particles 12. The microgel particles 12 are secured to one another at points of physical contact via annealing connections 13 as illustrated in FIG. 1A. FIG. 1A illustrates the microgel particles 12 having a spherical shape. However, it should be understood that the microgel particles 12 may have non-spherical shapes as well. The scaffold 10 includes interstitial spaces therein 14 that are voids that form micropores within the larger scaffold 10. The interstitial spaces 14 have dimensions and geometrical profiles that permit the infiltration, binding, and growth of cells and tissue from the surrounding tissue environment. It should be appreciated that the microporous nature of the scaffold 10 disclosed herein involves a network of interstitial spaces or voids 14 located between annealed microgel particles 12 that form the larger scaffold structure. FIG. 1A further illustrates a plurality of analyte-specific fluorophores 16 conjugated to the microgel particles 12. An analyte may include any molecule or chemical species that is desired to be measured. This may include biomolecules, organic molecules, inorganic molecules, drugs, drug metabolites, and the like. As explained further herein, the scaffold 10 is formed on or in tissue of a subject or patient. For example, the scaffold 10 may be formed in an excision that is made in skin tissue. Alternatively, the scaffold 10 may also be injected into or below the skin of the subject or patient. For example, the scaffold 10 may be injected subcutaneously. In yet another alternative, the three dimensional scaffold 10 may be applied in or on other tissue types or bodily organs different than skin.

According to one or more embodiments described herein, the analyte-specific fluorophores 16, in the presence of the analyte and subject to excitation radiation, emit fluorescent light. This fluorescent light is then captured or read using, for example, a reader device as described herein. The fluorescent light may include visible light as well as light in the non-visible spectrum (e.g., infrared light). In some embodiments, the intensity of the read fluorescent light is then used to calculate a concentration of the analyte based on the intensity. In other embodiments, a fluorescent pattern may be produced by the emitted fluorescent light and the pattern is used to determine the concentration of the analyte. Alternatively, in some embodiments, the reader device may not be needed as the fluorescent light or pattern may be visualized manually.

In one aspect of the subject matter described herein, the microporous gel system uses microgel particles 12 having diameter dimensions within the range from about 5 μm to about 1,000 μm. In one particular preferred aspect of the invention, the microgel particles 12 are substantially spherical in shape and having diameters within the range from about 30 micrometers to about 150 micrometers. The microgel particles 12 may be made from a hydrophilic polymer, amphiphilic polymer, synthetic or natural polymer (e.g., poly(ethylene glycol) (PEG), poly(propylene glycol), poly(hydroxyethylmethacrylate), hyaluronic acid (HA), gelatin, fibrin, chitosan, heparin, heparan, and synthetic versions of HA, gelatin, fibrin, chitosan, heparin, or heparan). In one embodiment, the microgel particles 12 are made from any natural (e.g., modified HA) or synthetic polymer (e.g., PEG) capable of forming a hydrogel. In one or more embodiments, a polymeric network and/or any other support network capable of forming a solid hydrogel construct may be used. Suitable support materials for most tissue engineering/regenerative medicine applications are generally biocompatible and preferably biodegradable. Examples of suitable biocompatible and biodegradable supports include: natural polymeric carbohydrates and their synthetically modified, crosslinked, or substituted derivatives, such as gelatin, agar, agarose, crosslinked alginic acid, chitin, substituted and cross-linked guar gums, cellulose esters, especially with nitrous acids and carboxylic acids, mixed cellulose esters, and cellulose ethers; natural polymers containing nitrogen, such as proteins and derivatives, including cross-linked or modified gelatins, and keratins; vinyl polymers such as poly(ethyleneglycol)acrylate/methacrylate/vinyl sulfone/maleimide/norbornene/allyl, polyacrylamides, polymethacrylates, copolymers and terpolymers of the above polycondensates, such as polyesters, polyamides, and other polymers, such as polyurethanes; and mixtures or copolymers of the above classes, such as graft copolymers obtained by initializing polymerization of synthetic polymers on a preexisting natural polymer. A variety of biocompatible and biodegradable polymers are available for use in therapeutic applications; examples include: polycaprolactone, polyglycolide, polylactide, poly(lactic-co-glycolic acid) (PLGA), and poly-3-hydroxybutyrate. Methods for making networks from such materials are well-known.

In one or more embodiments, the microgel particles 12 further include covalently attached chemicals or molecules that act as signaling modifications that are formed during microgel particle 12 formation. Signaling modifications includes the addition of, for example, adhesive peptides, extracellular matrix (ECM) proteins, and the like. Functional groups and/or linkers can also be added to the microgel particles 12 following their formation through either covalent methods or non-covalent interactions (e.g., electrostatic charge-charge interactions or diffusion limited sequestration). Crosslinkers are selected depending on the desired degradation characteristic. For example, crosslinkers for the microgel particles 12 may be degraded hydrolytically, enzymatically, photolytically, or the like. In one particular preferred embodiment, the crosslinker is a matrix metalloprotease (MMP)-degradable crosslinker.

Examples of these crosslinkers are synthetically manufactured or naturally isolated peptides with sequences corresponding to MMP-1 target substrate, MMP-2 target substrate, MMP-9 target substrate, random sequences, Omi target sequences, Heat-Shock Protein target sequences, and any of these listed sequences with all or some amino acids being D chirality or L chirality. In another embodiment, the crosslinker sequences are hydrolytically degradable natural and synthetic polymers consisting of the same backbones listed above (e.g., heparin, alginate, poly(ethyleneglycol), polyacrylamides, polymethacrylates, copolymers and terpolymers of the listed polycondensates, such as polyesters, polyamides, and other polymers, such as polyurethanes).

In another embodiment, the crosslinkers are synthetically manufactured or naturally isolated DNA oligos with sequences corresponding to: restriction enzyme recognition sequences, CpG motifs, Zinc finger motifs, CRISPR or Cas-9 sequences, Talon recognition sequences, and transcription factor-binding domains. Any of the crosslinkers from the listed embodiments are activated on each end by a reactive group, defined as a chemical group allowing the crosslinker to participate in the crosslinking reaction to form a polymer network or gel, where these functionalities can include: cysteine amino acids, synthetic and naturally occurring thiol-containing molecules, carbene-containing groups, activated esters, acrylates, norborenes, primary amines, hydrazides, phosphenes, azides, epoxy-containing groups, SANPAH containing groups, and diazirine containing groups.

In an alternative embodiment, the microgel crosslinker is non-biodegradable. For example, PEG dithiol, to allow for a long lasting sensing scaffold. Alternatively, the scaffold can be made to be degradable over time by incorporating MMP-degradable crosslinkers to allow resorbability of the sensor over time, or combinations of degradable and non-degradable microgels can be incorporated at varying stoichiometries (e.g. 1:1, 1:10, 1:20, 1:5) to maintain sufficient sensor material while allowing tissue to also regenerate surrounding the sensors. In still another embodiment, the microgel crosslinker may be specifically designed to photolytically degrade in response to an applied light (e.g., ultra violet light). In this regard, the microgel scaffold 10 that is formed may be used over a long lifetime at the site of application (e.g., months). As noted herein, it has been found experimentally that microgel scaffolds 10 that are not biodegradable tend to have better tissue ingrowth properties. Should the need arise to remove the microgel scaffold 10, the subject area may be illuminated with light for a period of time to accelerate and/or promote the breakdown of the microgel scaffold 10. Examples of crosslinkers that may be degraded with the application of light include macromers incorporating o-nitrobenzyl groups such as those disclosed in Griffin et al., Photodegradable macromers and hydrogels for live cell encapsulation and release, J. Am Chem Soc., 134(31), pp. 13103-7 (2012) as well as light degradable functionalities disclosed in Yanagawa et al., Partially photodegradable hybrid hydrogels with elasticity tunable by light irradiation, Colloids Surf B Biointerfaces, 126, pp. 575-9 (2015); and Shin et al., Photodegradable hydrogels for capture, detection, and release of live cells, Angew Chem Int Ed. Engl., 53(31), pp. 8221-4 (2014), all of which are incorporated herein by reference.

Although it is not expected that the PEG backbone is immunogenic, for long-term intradermal monitoring adaptive immune response to glucose sensing or other sensing moieties could develop, leading to signal decrease and drift. Covalent immobilization partially addresses this issue by preventing uptake by dendritic or other antigen presenting cells. In addition microgel precursors with embedded sensor moieties can be coated with additional hydrogel material that does not include these moieties and is more bio-inert, forming a core-shell structure. For example, in this embodiment, the microgel particles 12 would have an inner core that contains analyte-specific fluorophores that is surrounded by an outer shell of hydrogel material that has no analyte-specific fluorophores contained thereon. The hydrogel material of the outer shell may also consist of a different material such as that disclosed in Robitaille et al., Studies on small (<350 micron) alginate-poly-L-lysine microcapsules. V. Determination of carbohydrate and protein permeation through microcapsules by reverse-size exclusion chromatography, J Biomed Mater Res., 5; 50(3):420-7 (2000), which is incorporated by reference herein. The hydrogel material of the outer shell may also have increased cross-linking density (see Weber et al., Effects of PEG hydrogel crosslinking density on protein diffusion and encapsulated islet survival and function, J Biomed Mater Res A., 90(3): 720-729 (2009), which is incorporated by reference herein) to have a smaller pore size to prevent the diffusion of proteins or enzymes through the shell (e.g. oxidases or other enzymes that could degrade the fluorophores) while still allowing the free transit of small molecule analytes (e.g., glucose). This outer shell would also prevent antigen presenting cells from uptaking the potentially immunogenic fluorophore compounds. A core-shell structure can be created by reflowing the microgel precursors with sensor moieties into a microfluidic channel containing pre-polymer and forming a new droplet with embedded microgel that then fully polymerizes. Alternatively, polymerization or linkage (e.g. to form PEG) off of the surface can be conducted in a solution of microgel precursors, as long as the annealing linker is still present at the interface of this new core-shell microgel.

In one embodiment, the chemistry used to generate microgel particles 12 allows for subsequent annealing and scaffold formation through radically-initiated polymerization. This includes chemical-initiators such as ammonium persulfate combined with Tetramethylethylenediamine. Alternatively, photoinitators such as Irgacure® 2959 or Eosin Y together with a free radical transfer agent such as a free thiol group (used at a concentration within the range of 10 μM to 1 mM) may be used in combination with a light source that is used to initiate the reaction as described herein. One example of a free thiol group may include, for example, the amino acid cysteine, as described herein. Of course, peptides including a free cysteine or small molecules including a free thiol may also be used. Another example of a free radical transfer agent includes small molecules presenting vinyl moieties, such as N-Vinylpyrrolidone (NVP).

Alternatively, Michael and pseudo-Michael addition reactions, including α,β-unsaturated carbonyl groups (e.g., acrylates, vinyl sulfones, maleimides, and the like) to a nucleophilic group (e.g., thiol, amine, aminoxy) may be used to anneal microgel particles 12 to form the scaffold. In another alternative embodiment, microgel particle formation chemistry allows for network formation through initiated sol-gel transitions including fibrinogen to fibrin (via addition of the catalytic enzyme thrombin).

Functionalities that allow for particle-particle annealing are included either during or after the formation of the microgel particles 12. In one or more embodiments, these functionalities include α,β-unsaturated carbonyl groups that can be activated for annealing through either radical initiated reaction with α,β-unsaturated carbonyl groups on adjacent particles or Michael and pseudo-Michael addition reactions with nucleophilic functionalities that are either presented exogenously as a multifunctional linker between particles or as functional groups present on adjacent particles. This method can use multiple microgel particle 12 population types that when mixed form a scaffold 10. For example, microgel particle of type X presenting, for example, nucleophilic surface groups can be used with microgel particle type Y presenting, for example, α,β-unsaturated carbonyl groups. In another embodiment, functionalities that participate in Click chemistry can be included allowing for attachment either directly to adjacent microgel particles 12 that present complimentary Click functionalities or via an exogenously presented multifunctional molecule that participates or initiates (e.g., copper) Click reactions.

The annealing functionality can include any previously discussed functionality used for microgel crosslinking that is either orthogonal or similar (if potential reactive groups remain) in terms of its initiation conditions (e.g., temperature, light, pH) compared to the initial crosslinking reaction. For example if the initial crosslinking reaction consists of a Michael-addition reaction that is temperature dependent, the subsequent annealing functionality can be initiated through temperature or photoinitiation (e.g., Eosin Y, Irgacure®). As another example, the initial microgels may be photopolymerized at one wavelength of light (e.g., ultraviolent with Irgacure®), and annealing of the microgel particles 12 occurs at the same or another wavelength of light (e.g., visible with Eosin Y) or vice versa. Besides annealing with covalent coupling reactions, annealing moieties can include non-covalent hydrophobic, guest/host interactions (e.g., cyclodextrin), hybridization between complementary nucleic acid sequences or nucleic acid mimics (e.g., protein nucleic acid) on adjoining microgel particles 12 or ionic interactions. An example of an ionic interaction would consist of alginate functionality on the microgel particle surfaces that are annealed with Ca2+. So-called “A+B” reactions can be used to anneal microgel particles 12 as well. In this embodiment, two separate microgel types (type A and type B) are mixed in various ratios (between 0.01:1 and 1:100 A:B) and the surface functionalities of type A react with type B (and vice versa) to initiate annealing. These reaction types may fall under any of the mechanisms listed herein.

A variety of sensing modalities can be incorporated into the microgel particles 12, including fluorescence intensity measurements, fluorescent pattern, or fluorescence lifetime-based sensor that relates to the time-course of fluorescent emission from the sensor. In one particular embodiment, Concanavalin A (Con A)-based fluorophores 16, or glucose-sensitive fluorophores 16 based on diboronic or arylboronic acids which are known glucose sensor moieties can be incorporated into or onto the microgel particles 12. Concanavalin A (Con A) is a plant-sugar-binding protein that binds to mannose and glucose. Con A has four receptor sites that allow reversible glucose binding. Glucose competes with fluorescently labeled dextran for binding to Con A. When fluorescein-labeled dextran binds to Con A, the charge transfer quenches (i.e., reduces) fluorescence intensity. Glucose, however, preferentially binds to Con A and displaces fluorescein-dextran. Because of this, in the presence of glucose, the emitted light from the free dextran molecules increases. Another sensing modality that may be used is glucose oxidase and an oxygen-sensitive fluorophore 16. The boronic acid moiety may also be used for detection of sugars due to the high sensitivity and reversible binding in aqueous conditions. Separate UV-excited fluorophores Mellitus Blue™ glucose probes and Mellitus Violet™ glucose probes (Ursa Biosciences LLC, Bel Air, MD) also are sensitive to glucose concentrations and decrease in intensity with higher concentrations. A variety of different sensing modalities known to those skilled in the art may be used with this invention. These include the sensing schemes discussed in Heo et al., Towards Smart Tattoos: Implantable Biosensors for Continuous Glucose Monitoring, Adv. Healthcare Mater., 2, 43-56 (2013), which is incorporated by reference herein.

Traditionally, fluorophores that are excited in near infrared are used because of the relative transparency of tissue in this regime, however, for one preferred embodiment of the device described herein in which sensors are placed “intradermally,” tissue transparency is less of a concern and a broader range of fluorophores may be used for sensing, including fluorophores excited by UV and blue light. Alternatively, glucose-binding engineered fluorescent proteins or glucose sensing aptamers linked to fluorescent aptamer structures such as Spinach aptamer could be incorporated.

Linkages of fluorophores 16 within the PEG hydrogel polymer structure of the microgel particles 12 can be performed through a variety of chemistries to, for example, free vinylsulfone (VS) groups on the PEG-VS backbone, or through photo-caging techniques to sequester small molecules. Spacer arms linking the sensor moiety to the microgel PEG matrix, consisting of a water soluble polymer chain (e.g., MW 3400 PEG) to allow mobility of the moiety can also increase the signal. Reference fluorophores known in the art that are not sensitive to glucose (but may be sensitive to temperature, oxygen concentration, or pH) can allow for calibration of pH and other effects on the glucose sensing moiety to get improved accuracy measurements and calibration. Ideally, such an approach can reduce the calibration interval to less than twice daily.

FIG. 1B illustrates the analyte sensing scaffold 10 that is formed intradermally within the skin 100 tissue of a subject or patient. The analyte sensing scaffold 10 illustrated in FIG. 1B is annealed or cross-linked via annealing connections 13 as illustrated in FIG. 1A. In this embodiment, cells are able to pass through porous interstitial spaces 14 (best seen in FIG. 1A) formed between the microgel particles 12. The analyte sensing scaffold 10, post-healing, is also exposed to bodily fluids of the subject or patient such that the analyte of interest is able to come into physical contact with the analyte-specific fluorophores 16 conjugated to the microgel particles 12 as seen in FIG. 1A. As explained below, the presence and concentration of particular analytes may be detected and measured in response to exposure to an excitation light source using, for example, an optional optical readout device 30.

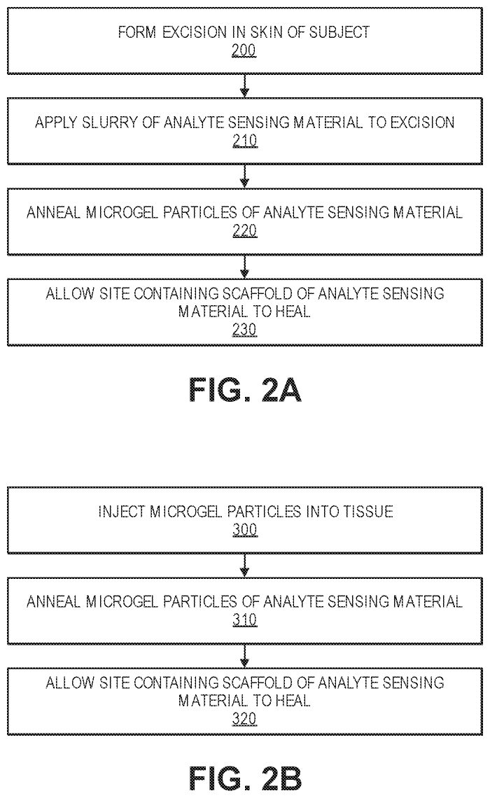

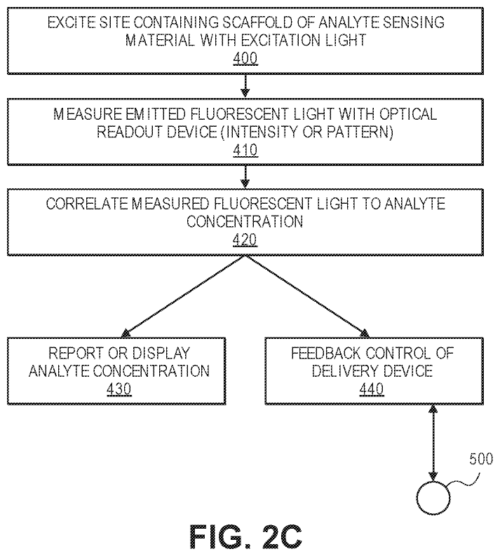

FIG. 2A illustrates a flowchart of a typical procedure that is used to form the three dimensional scaffold 10 structure using the plurality of annealed microgel particles 12. First, as seen in operation 200 a patient or subject comes to a medical facility such as a clinic, medical office, or hospital setting where a small full-thickness excision is made in the skin. The excision may be formed using a scalper, knife, or other surgical tool typically used by physicians. A “full-thickness” excision is involves removing skin through deeper dermal layers, down to the fatty layer. The full-thickness excision does not need to be large; for example an excision that is between 3-5 mm in diameter may be made although the particular size of the excision is not critical. The full-thickness excision may be made just prior to the addition of the microgel particles 12. Next, as seen in operation 210, a slurry of the analyte sensing material (i.e., microgel particles 12) is applied to fill the excision from an applicator such as a single-use applicator (e.g. syringe applicator). As explained herein, the analyte sensing material includes a collection of microgel particles 12 having one or more network crosslinker components. An endogenous or exogenous (e.g., located in the applicator device) annealing agent then links the microgel particles 12 together in situ to form a covalently-stabilized scaffold of microgel particles 12 having interstitial spaces therein. The annealing agent may include, for example, Factor XIIIa, Eosin Y, a free radical transfer agent, or some combination thereof. The annealing agent may also be present at the delivery site of the microgel particles 12 (i.e., endogenous annealing agent). For analyte sensing, a plurality of analyte-specific fluorophores 16 are conjugated to the microgel particles 12, wherein the analyte-specific fluorophores 16, in the presence of the analyte and subject to excitation radiation, emit fluorescent light. After application of the microgel particles 12 and optional annealing agent, the microgel slurry is annealed as seen in operation 220. In some embodiments, annealing may take place automatically after delivery of the microgel particles 12 and optional annealing agent to the delivery site. Alternatively, in some embodiments, an external stimulus such as light is applied to initiate cross-linking. For example, the light could be delivered via a wide spectrum white light (incandescent or LED), or a green or blue LED light. A flashlight, wand, lamp, or even ambient light may be used to supply the white light. Exposure should occur between 0.1 seconds and 300 seconds, and the intensity of light should range between 0.1 mW/cm² to 100 mW/cm² at the site of annealing. In one embodiment, light is exposed to the applied microgel particles 12 (e.g., with 30 seconds of white light exposure) to form the contiguous scaffold that is linked to the underlying tissue. Next, as seen in operation 230, the area is bandaged and allowed to heal over 14-21 days until significant dynamic tissue changes are no longer occurring within or around the sensing scaffold. In the specific embodiment that utilizes UV photodegradable linkers (i.e. o-nitrobenzyl groups); using white light or visible light is advantageous to prevent degradation of microparticle gels during cross-linking of microgel particles 12.

FIG. 2B illustrates a flowchart of an alternative procedure that is used to form the three dimensional scaffold 10 structure using the plurality of annealed microgel particles 12. In this alternative procedure, instead of forming an excision in the skin and applying a slurry thereto, the microgel particles 12 are injected into tissue as seen in operation 300. An injection tool such as a syringe or the like can be used to inject the microgel particles 12 into, within, or under a body tissue. The body tissue may include, for example, skin tissue. In one example, the injection tool may be a tattoo gun or similar implement that can create a pattern or graphic containing the microgel particles 12. As seen in FIG. 2B, after the microgel particles 12 have been injected, the scaffold is formed as described above with respect to the embodiment of FIG. 2A. Namely, the microgel particles 12 are then annealed to form the scaffold 10 of analyte sensing material as seen in operation 310. The delivery site is then allowed to heal for a period of time as illustrated in operation 320.

FIG. 2C illustrates a flowchart showing the process of illumining the scaffold containing the microgel particles 12 for analyte detection and concentration measurement. As seen in FIG. 2C, the tissue site containing the analyte sensing scaffold 10 is excited with excitation light as seen in operation 400. The excitation light may include light of a particular wavelength (or multiple wavelengths) that are known to excite the conjugated fluorophores 16 on the microgel particles 12. This may include a narrow band light source such as those emitted by LEDs or laser diodes. Appropriate filters may be used to tailor the excitation wavelength to the appropriate wavelength range to produce fluorescence. Next, as seen in operation 410 of FIG. 2C, the emitted fluorescent light is then read using an optional optical readout device 30. The optical readout device 30 contains an image sensor such as a CCD sensor, CMOS sensor, or photodiode and detects and measures the intensity of the fluorescent light that is emitted from the excited fluorophores with a high sensitivity detector.

In one embodiment, the optical readout device 30 measures the overall or average intensity of the emitted fluorescent radiation. In another embodiment, the optical readout device 30 captures a pattern of fluorescent light that is used to determine the concentration of the analyte (explained in more detail below). The measured intensity value (or pattern) is then correlated to a concentration of the analyte as seen in operation 420. For example, a calibration curve or calibration function can be created and optionally stored in the optical readout device 30 and used to correlate measured intensity to analyte concentration. In some embodiments, increased concentration of the analyte will result in increased fluorescence. In other embodiments, increased concentration of an analyte will result in decreased fluorescence. In another embodiment, the scaffold is made with a pattern or gradient containing a varied concentration of fluorophores. The detected pattern image may be detected by the optical readout device 30 and the resulting pattern used to correlate to a specific analyte concentration. In this particular embodiment where a pattern is used that changes pattern shape or size based on the concentration of analyte present the optical readout device 30 may, in some embodiments, be omitted entirely as the pattern may be able to be viewed with the naked eye after illumination with excitation light.

Still referring to FIG. 2C, the concentration of the analyte may then be reported out to the user and/or displayed to the user using the optical readout device 30 as illustrated in operation 430. The optical readout device 30 may thus contain a display therein that is used to visually display the concentration of the analyte. Alternatively, the concentration may be reported out aurally to the patient. In some embodiments, the readout device 30 may also communicate with other personal electronic devices (e.g., Smartphone, tablet computer, stand-along reader device) that can be used to read and track the measured concentration of the analyte.

In some embodiments, the concentration of the analyte may be communicated to a drug delivery device 500 that is used to deliver a drug, medicament, or pharmaceutical to the patient as is illustrated in operation 440. This communication may be a signal or data that is communicated wirelessly to the drug delivery device 500 which is used for feedback purposes. The transmission of the analyte concentration to the drug delivery device 500 may be an alternative to, or in addition to, the reporting or display of the analyte concentration. For example, the concentration of the analyte may be input to a drug delivery device in the form of a pump that is worn or implanted in the subject. The concentration of the analyte is then used as a feedback input in the drug delivery device 500 to control the delivery of the drug to the patient. For example, the drug delivery device 500 may be an insulin pump that is used for diabetic patients. The reported concentration of glucose can be sent to the insulin pump or control circuitry controlling the same which can then be used to control the timing and amount of insulin that is delivered to the subject.

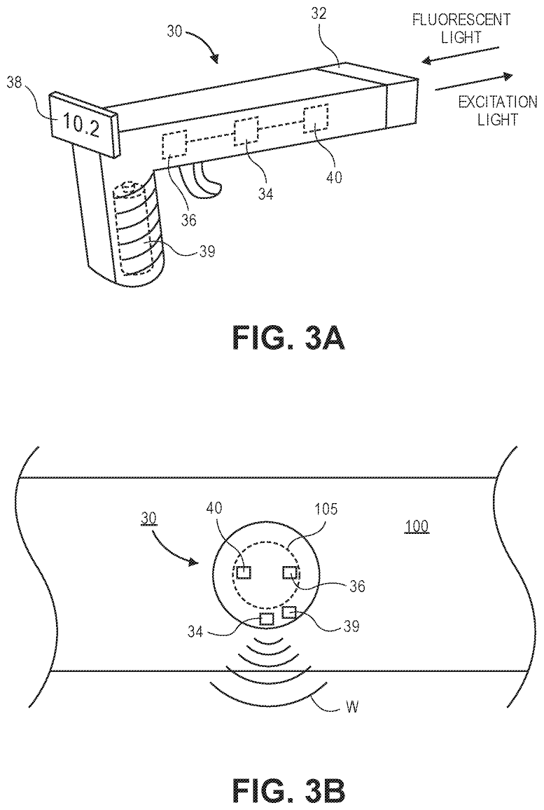

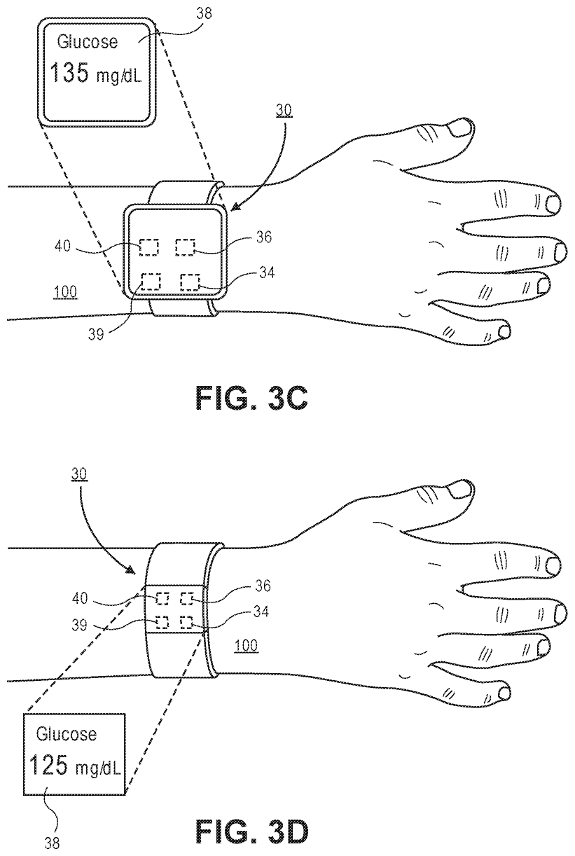

The excitation light source 40 may be located in a separate device from the optical readout device 30. In some embodiments, there may be multiple different light sources 40, with each source designed for a particular fluorophore 16. Alternatively, the optical readout device 30 may also include the excitation light source(s) 40. The optical readout device 30 may optionally also include one or more filters that are placed snugly over the sensing region to block out ambient light or excitation light. The optical readout device 30 may also include a hand-held device e.g., a gun as illustrated in FIG. 3A that is pressed against the skin 100 of the subject at the location of the site where the scaffold 10 of analyte sensing material has been placed. As seen in FIG. 3A, the gun-type optical readout device 30 includes a shroud 32 that can be pressed against the skin 100 of a subject so that ambient light does not interfere with the fluorescent measurements. In this embodiment, the gun-type optical readout device 30 includes the excitation light source 40 therein that is powered by control circuitry 34. Control circuitry may be used to pulse the excitation light source 40 in short pulses (e.g., approximately 1 ms) to avoid bleaching as discussed herein. A detector 36 such as an image sensor is also located in the optical readout device 30 and is coupled to control circuitry 34 that is configured to read the intensity and/or pattern of the fluorescent light emitted by the scaffold 10 of analyte sensing material. The optical readout device 30 includes a display 34 that is used to report the concentration of the analyte to the user. A power source 39 such as a battery is provided in the optical readout device 30 for powering the excitation light source 40, circuitry 34, and detector 36. Power could also be provided by an external power source.

The optical readout device 30 may also take a number of different forms as seen in FIGS. 3B-3D and could be worn comfortably under clothes. FIG. 3B illustrates the optical readout device 30 in the form of a patch or bandage that is placed over the excision site 105. The patch or bandage version of the optical readout device 30 has an excitation light source 40 contained therein along with a fluorescent light detector 36 and control circuitry 34. A battery 39 is integrated into the patch or bandage to provide power to the various components. In this embodiment, data may be transmitted wirelessly W through known wireless protocols (e.g., Bluetooth, Wi-Fi, near field communication) to remote device that can be used to display the measured concentration of the analyte. For example, a Smartphone, tablet PC, or stand-alone reader device may be used to receive the wireless signal and display or otherwise report the measured concentration. The wireless signal W may also be used to communicate with a drug delivery device 500 such as that illustrated in FIG. 2C.

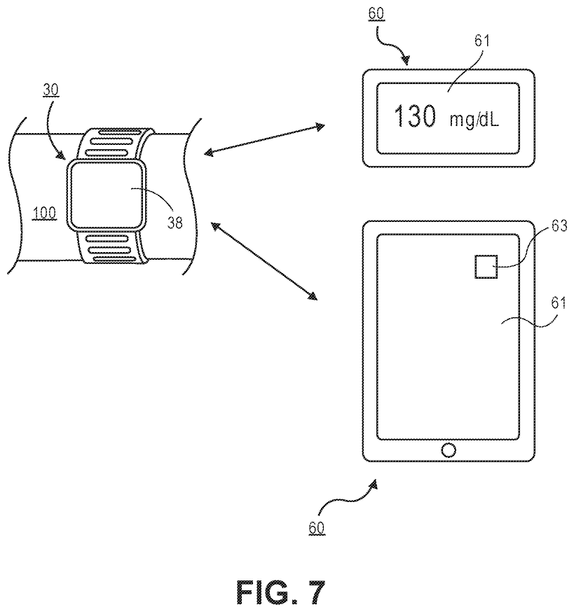

The optical readout device 30 could also be a watch as illustrated in FIG. 3C and FIG. 7. FIG. 3C illustrates a watch version of the optical readout device 30 that includes an excitation light source 40 contained therein along with a fluorescent light detector 36 and control circuitry 34. A battery 39 is integrated into the watch to provide power to the various components. One side of the optical readout device 30 is pressed against the surface of the skin 100 of a subject that contains the excision site 105 that contains the scaffold 10 of analyte sensing material. The watch version of the optical readout device 30 contains a display 38 which can be used to display the measured concentration of the analyte. In the particular embodiment of FIG. 3C, glucose is the analyte that is measured using the scaffold 10 of analyte sensing material and the measured concentration is displayed to the user on the display 38. The optical readout device 30 may report the measured concentration periodically to the user, for example, programmed to a specific schedule. The optical readout device 30 may also be controlled by the user so that the concentration can be measured at particular times chosen by the user (e.g., before or after meals). Alternatively, the optical readout device 30 may continuously monitor the concentration of the analyte over shorter intervals of seconds, minutes, or hours.

FIG. 3D illustrates another embodiment of an optical readout device 30 that is in the form of a band or wrap that includes an excitation light source 40 contained therein along with a fluorescent light detector 36 and control circuitry 34. A battery 39 is integrated into the band or wrap device to provide power to the various components. The optical readout device 30 may contain a display 38 on the band or wrap device that is used to display readings to the user as is explained above in the context of the embodiment of FIG. 3C.

One of the challenges with fluorophore-based sensors is the bleaching of fluorophores over time which leads to drift in signal and reduction in sensitivity. This can be partially addressed with the design and engineering of the optical readout device 30, for example using techniques such as pulsed excitation for approximately 1 ms and rapid readout with sensitive photodetectors to prevent bleaching induced drift. In certain embodiments, the optical readout device 30 may seal over the site of an intradermally-placed scaffold 10 of analyte sensing material and blocks out ambient light.

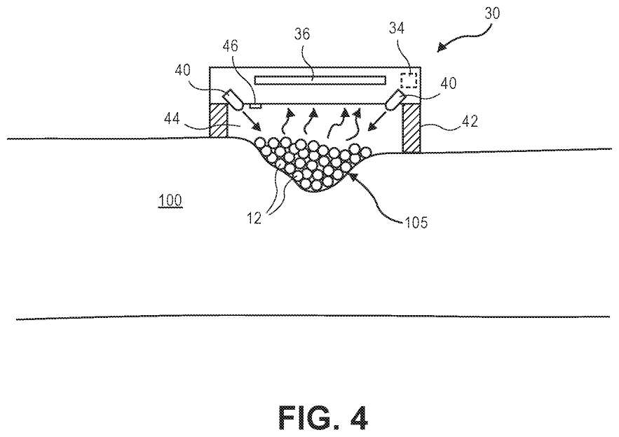

FIG. 4 illustrates one embodiment of an optical readout device 30 that includes light sources 40 (e.g., light emitting diodes (LEDs)) for emitting excitation radiation that excites the fluorophores 16 within the analyte sensing scaffold 10 of microgel particles 12. The fluorescent light that is emitted is detected by the fluorescent detector 36 (e.g., high sensitivity CCD or CMOS chip). In one aspect of the invention, the intensity value of the imaged region (e.g., average intensity) is then used to generate a concentration of the analyte based on, for example, a calibration curve or calibration functions. A processor or control circuitry 34 located in the optical readout device may run software or an application or “app” that then is able to convert the measured fluorescent value to a concentration using the pre-stored calibration curve or function. The calculated or determined concentration of the analyte may then be presented to the user on a display 38 associated with the optical readout device 30 in certain embodiments (e.g., as seen in FIGS. 3C, 3D, and 7). Alternatively, the raw image files or the intensity value(s) can be transmitted to a remote display device 60 as seen in FIG. 7 (either wired or wirelessly through Bluetooth, Wi-Fi, near field communication or other wireless communication protocol) whereby the remote display device 60 may output the analyte concentration. In one aspect, the remote display device 60 may include a separate standalone display device that receives data and/or images transmitted from the optical readout device 30. Alternatively, the remote display device 60 may include, for example, a portable electronic device such as Smartphone, tablet PC, or the like that runs the software or an application that is used to display readings to a user. The data or images may be communicated via a wired or wireless connection to the remote display device 60 using, for example, Bluetooth, Wi-Fi, near field communication or other wireless protocol.

In the embodiment of FIG. 4, a light seal 42 is formed around the periphery of the optical readout device 30 and may take the form of a wall or raised surface that creates a dark cavity or recess 44 that is substantially isolated from ambient light. In one embodiment the dark cavity or recess 44 of the optical readout device 30 in which excitation and readout occurs also includes an ambient light sensor 46 that when above or below a threshold value informs the user if the optical readout device 30 is well registered and sealed against the skin 100. The light sensor 30 may also be used in calibration. In the embodiment of FIG. 4, the optical readout device 30 may contain fasteners (e.g., bands or ties), wrapping, and/or adhesive elements so that the optical readout device 30 can be worn continually, e.g. around the upper or lower arm, or against the sternum or hip region. The optical readout device 30 may contain one or more filtered light sources to excite fluorescence within the analyte sensing scaffold 10 for interrogating sensing moieties (e.g., glucose) and other calibration/sensing moieties. In some embodiments the optical readout device 30 may contain one or more lenses that are used to focus emitted fluorescent light onto the fluorescent detector 36. Optional emission filters may be included to filter the emitted fluorescent light or filter out scattered light from the excitation light source 40. Of course, the use of lenses is optional as the optical readout device 30 may also function without any lenses or focusing optics. Spatial localization of fluorescence by the optical readout device 30 may be useful in some embodiments and can be achieved with an imaging sensor or position sensitive detector.

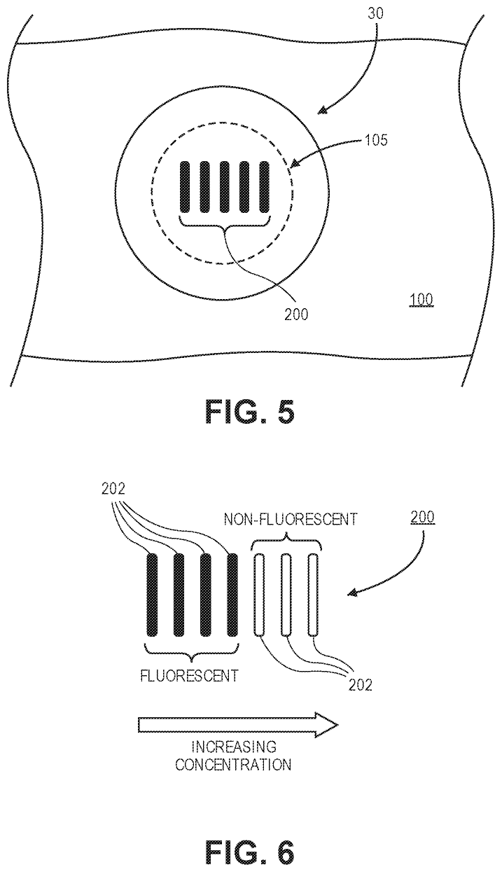

FIG. 5 illustrates an embodiment of an optical readout device 30 that is placed on the skin 100 of the subject at the location of the excision 105 containing the analyte sensing scaffold 10 in a pattern 200. The optical readout device 30 is able to image or detect the fluorescent pattern 200 that is created by the analyte sensing scaffold 10 in response to exposure to the analyte. In this embodiment, the fluorescent pattern 200 that is created is used to determine the concentration of the analyte of interest. FIG. 5 illustrates an embodiment whereby a series of lines fluoresce in response to contact with the analyte. The size or number of lines that fluoresce can be used to determine the concentration of the analyte. For example, FIG. 6 illustrates a pattern 200 whereby the number of fluorescent lines 202 is used as a proxy to determine the concentration of the analyte. For example, if only one or a few lines 202 fluoresce then the concentration of the analyte may be read as low (or translated into a specific concentration value or range of concentration values). Conversely, if most or all of the lines 202 fluoresce this may translate into a reading of a high concentration of the analyte (or translated into a specific concentration value or range of concentration values). For example, different lines 202 may have different amounts of fluorophores 16 conjugated to the microgel particles 12. Lines 202 with a lower level of loading of fluorophores 16 or fluorophores with lower affinity to analyte may require a higher level of analyte to fluoresce at detectible levels. This type of differential loading of the microgel particles 12 may be used to create a pattern that can be used to determine analyte concentration.

FIG. 7 illustrates an embodiment in which the optical readout device 30 communicates with a remote display device 60. The remote display device 60 may communicate with the optical readout device 30 wirelessly using known wireless communication profiles (e.g., Wi-Fi, Bluetooth, near field communication, etc.). In some embodiments, information may be displayed to the user on only the remote display device 60 thus permitting the optical readout device 30 to be small in size. In other embodiments, information may be displayed on both the display 38 associated with the optical readout device 30 as well as the display 61 associated with the remote display device 60. As seen in FIG. 7, in some embodiments, the remote display device 60 may include a mobile communication device such as a Smartphone. The Smartphone includes a display 61 that can be used to display information to the user. The Smartphone may also contain a software application or “app” 63 that is used to communicate and interact with the optical readout device 30. In this regard, in certain embodiments, two-way communication between the remote display device 60 and the optical readout device 30 is possible.

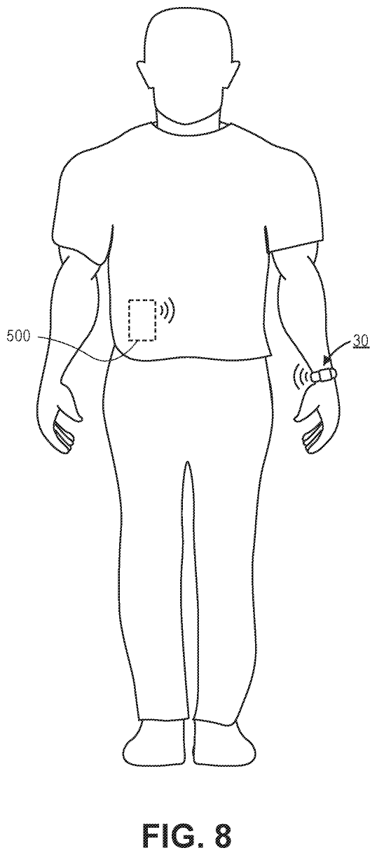

FIG. 8 illustrates another embodiment of a system in which the optical readout device 30 communicates directly or indirectly to a drug delivery device 500 such as a pump that is used to deliver a drug to the subject. In this way, depending on level of analyte measured by the optical readout device 30, the drug delivery device 500 can be controlled to deliver, stop delivering, or alter the rate of delivery of a drug to the subject. Communication to the drug delivery device 500 may be accomplished wirelessly as described herein. For example, the drug delivery device 500 may receive the latest concentration of the analyte as measured by the optical readout device 30 and then adjust the delivery rate accordingly based on the measured concentration. For example, the drug delivery device 500 may be an insulin pump that is worn by the subject or implanted internally. The insulin pump may receive periodic transmissions from the optical readout device 30 (e.g., every 15 minutes) of the measured glucose levels. Based on the measured glucose levels the insulin pump may deliver a bolus or quantity of insulin to the patient to control the glucose level within the subject to optimally treat a patient or subject with diabetes.

In another embodiment, the analyte sensing scaffolds 10 (e.g., 1-3 mm in diameter) with and without sensor moieties (e.g., fluorophores 16) can be placed adjacent to each other and the signal from the analyte sensing scaffold without the sensor moieties can be used to normalize the signal from the analyte sensing sensor scaffold 10 containing the sensor moieties. Alternatively, a mixture of microgel particles 12 with and without sensor moieties (e.g., fluorophores 16) are poorly mixed to yield a mosaic pattern of sensor/non-sensor microgel regions (e.g. not evenly mixed) that have a unique spatial distribution, fingerprint, or pattern which is imaged and used for normalization/analysis by the optical readout device 30. FIGS. 5 and 6 illustrate one such pattern that uses bars or lines. At higher concentrations of the analyte additional bars or lines fluoresce. The concentration may be obtained by counting the number of illuminated bars or lines. This, of course, would require that the microgel scaffold 10 be created with such a pattern within the excision or otherwise injected into the skin tissue like a tattoo. For example, the pattern could be implanted into the skin using different microgel particle solutions containing different amounts of conjugated fluorophores 16.

It should be noted, that although some of the descriptions above refer to analyte sensing scaffolds 10 and optical readout devices 30 for glucose sensing, that the analyte sensing scaffold 10 and readout devices/systems can be combined with other sensing moieties embedded in microgel particles 12 to readout any blood metabolite one at a time or in a multiplexed manner. The properties of the sensing moiety should preferably be stable at body temperature, refreshable (i.e., binding to the metabolite is reversible), and water soluble or able to be made soluble in water when linking to hydrophilic linkers. An example of a general-purpose sensing moiety with these properties are the Spinach aptamer fusions such as those disclosed in Strack et al., Using Spinach-based sensors for fluorescence imaging of intracellular metabolites and proteins in living bacteria, Nature Protocols, 9(1), 146-155 (December, 2013), which is incorporated by reference herein. These fusions have been made sensitive to the small molecules adenosine, ADP, S-adenosylmethionine (SAM), guanine and GTP, and the proteins streptavidin, thrombin and MS2 coat protein (MCP). Spinach-based sensors for the second messengers cyclic di-GMP and cyclic AMP-GMP have also been reported. In addition to fluorescence intensity, fluorescence lifetime, fluorescence resonance energy transfer, and phosphorescence can also be used as sensing modalities, with specific dyes and fluorophores known in the art.

Oxygen, for example, is an important blood analyte that can report on peripheral artery disease, status of chronic wounds (diabetic ulcers, pressure sores), and reconstructive surgery. Oxygen generally quenches fluorescence from a broad range of fluorophores 16, however sensing fluorophores 16 that are particularly suited to this application include e.g. tris (4,7-diphenyl-1,10-phenanthroline) ruthenium (II) dichloride (GFS Chemicals, Powell, Ohio), Pt(II) meso-tetra(pentafluorophenyl) porphine (PtTFPP) (Frontier Scientific, Logan, Utah). Oxygen sensitive fluorophores are conjugated during the initial microgel cross-linking reaction to a fraction of the reactive groups on the 4-arm PEG (e.g. to vinylsulfone groups) using similar approaches as described in Griffin et al. Nat Materials 2015, which is incorporated by reference herein. This approach leads to a very high density of fluorophores available for reaction throughout the hydrogel matrix. Since the size of each microgel particle 12 in the linked scaffold of a plurality of microgel particles 12 is small compared to molecular diffusion length scales, analytes can easily reach and interact with the sensing fluorophores. The ratios of fluorophore per 4-arm PEG may be 1:4 to 1:100 while still maintaining sufficient vinylsulfone groups to enable microgel cross-linking and incorporation of cell adhesive peptides. Alternatively, smaller polymer microgel particles (e.g. co-polymer of styrene and pentafluorostyrene linked to these fluorophores through Click chemistry can be suspended and fixed in place in the larger microgel particles 12 following cross-linking. An example of such Click chemistry may be found in Koren et al., Stable optical oxygen sensing materials based on click-coupling of fluorinated platinum(II) and palladium(II) porphyrins—A convenient way to eliminate dye migration and leaching, Sens Actuators B. Chem, 169(5), pp. 173-81 (2012), which is incorporated by reference herein. Oxygen insensitive fluorophores 16 such as Nile blue can be incorporated alongside these oxygen sensing fluorophores 16 to calibrate for the optical system, scattering and absorbance of light by the tissue, etc. One exemplary embodiment includes degradable or non-degradable microgel particles 12 that are linked with an oxygen sensitive fluorophore and a reference fluorophore that are flowed into and annealed to fill a diabetic ulcer in order to provide tissue support and regenerative healing and simultaneously monitor regrowth of blood vessels and level of ischemia in the ulcer through the sensed level of oxygen.

In some embodiments described herein, the stiffness of the microgel scaffold 10 may be tuned or adjusted. In addition, the nature of the crosslinker that is used can determine whether the microgel scaffold 10 is biodegradable or non-biodegradable. In still other embodiments, which the microgel scaffold 10 may not be biodegradable it may still nonetheless be degradable through the application of light to the site of application. Moreover, it has been discovered that microgel scaffolds with increasing stiffness with non-biodegradable crosslinkers have better tissue intrusion properties.

In this experiment, microgel particles 12 were formed using 4-arm poly(ethylene glycol) vinyl sulfone (PEG-VS) backbone (20 kDa) that has been pre-modified with oligopeptides for cell adhesive properties (e.g., RGD) and surface/tissue annealing functionalities (e.g., K and Q peptides) along with a crosslinker. Microgel particles 12 were formed using a multi-inlet droplet generation device such as that disclosed in U.S. Patent Application Publication No. 2016/0279283, which is incorporated by reference herein. Generally, a first inlet is used to deliver the PEG-VS backbone that is functionalized while a second inlet is used to deliver the crosslinker while a third inlet delivers unmodified PEG-VS to prevent upstream mixing of the reagents relative to the droplet generation region.

The PEG-VS backbone may be prefunctionalized with 0.25 mM K-peptide (Ac-FKGGERCG-NH₂ [SEQ ID NO: 1]) (Genscript), 0.25 mM Q-peptide (Ac-NQEQVSPLGGERCG-NH₂ [SEQ ID NO: 2]), and various concentration (0.5 mM and 2.5 mM) of RGD (Ac-RGDSPGERCG-NH₂ [SEQ ID NO: 3]) (Genscript). The solution input to the first inlet may contain about 5% (on a weight basis) modified PEG-VS contained in a buffer of 0.3 M triethanolamine (Sigma), pH 8.25. The second inlet is coupled to a solution containing the crosslinker, which in one embodiment, is an 12 mM di-cysteine modified Matrix Metallo-protease (MMP) (Ac-GCRDGPQGIWGQDRCG-NH₂ [SEQ ID NO: 4] substrate (Genscript). In another embodiment, where the crosslinker is non-biodegradable the crosslinker that was used was PEG dithiol (MW 1,000). The third inlet is coupled to an aqueous solution containing 5% by weight of PEG-VS (unmodified by K, Q, or RGD peptides). A fourth inlet is used to deliver an oil phase that contains a surfactant (e.g., 1% SPAN® 80 by volume although other surfactants can be used). The contents of the droplets undergo mixing and will form the microgel particles 12 upon gelation, which in this embodiment is a function of the ambient temperature and the passage of time.

As used herein, K-peptides refer to those peptides that contain therein a Factor XIIIa recognized lysine group. As used herein, Q-peptides refer to those peptides that contain therein a Factor XIIIa recognized glutamine group. Thus, peptide sequences beyond those specifically mentioned above may be used. The same applies to the RGD peptide sequence that is listed above. All solutions can be sterile filtered through a 0.2 μm Polyethersulfone (PES) membrane in a Luer-lock syringe filter.

The microgel particles 12 that were generated were then extracted from the oil phase using either centrifugation through an aqueous phase or filtration through a solid membrane. The microgel particles 12 are then mixed with an annealing agent to anneal the microgel particles 12 to one another to form the three dimensional scaffold 10. In the experiments described herein, the microgel particles 12 were mixed with thrombin (2 U/mL) and FXIII (10 U/mL). Upon mixing, the thrombin activates the FXIII to form FXIIIa and the resulting FXIIIa is then responsible for annealing and linking of the K and Q peptides on adjacent microgel particles 12.

In experiments, the microgel particles 12 were spiked with Mesenchymal Stem Cells (MSCs) that were derived from the bone marrow of C57BL/6 mice and transfected with a lentiviral construct containing a Green Fluorescent Protein (GFP) expression motif. The microgel particles doped with FXIII and thrombin enzymes were spiked with MSCs at a concentration of 5,000 cells/μL. The spiked gels were then injected subcutaneously into mice using syringes. The mice were sacrificed at a scheduled point of time (56 days) and tissue samples were collected and in OCT medium for further analysis. Table 1 below illustrates the various scaffold materials 10 that were formed. As seen in Table 1, the stiffness of the scaffold 10 was adjusted as well as the type of crosslinker that was used (biodegradable or non-biodegradable).

TABLE 1

| | Storage | | | | | | | |
|---|---|---|---|---|---|---|---|---|
| | Modulus (after | | RGD | PEG | | Crosslinker | Crosslinker | |
| | swollen) | | Concentration | Concentration | | Type | Concentration | |
| Soft degradable | ~ 400 | Pa | 0.5 mM | 5 | wt. % | MMP | 4 | mM |
| | | | | | | degradable | | |
| Soft non- | ~ 450 | Pa | 0.5 mM | 4 | wt. % | Non- | 3 | mM |
| degradable | | | | | | biodegradable | | |
| Stiff 1 RGD | ~ 2,600 | Pa | 0.5 mM | 10 | wt. % | Non- | 9.9 | mM |
| | | | | | | biodegradable | | |
| Stiff 5 RGD | ~ 2,600 | Pa | 2.5 mM | 12 | wt. % | Non- | 11 | mM |
| | | | | | | biodegradable | | |

As seen above, microgel particles 12 with different degradability, stiffness, and RGD peptide (cell adhesion motif) concentration were subcutaneously injected. Preliminary data shows that non-degradable stiff gels (2.6 kPa) with moderate RGD concentration (0.5 mM) have the highest tissue ingrowth rate over longer periods of time important for implantable sensing (e.g., two months). Advantageously, the microgel scaffold 10 described herein permits cell and tissue ingrowth and avoids a sustained inflammatory response that would otherwise lead to the formation of a fibrous capsule around the implanted material. This microgel scaffold 10 incorporates one or more analyte sensing fluorophores 16 which are then able to be read by an optical readout device 30 as explained herein.

While embodiments of the present invention have been shown and described, various modifications may be made without departing from the scope of the present invention. The invention, therefore, should not be limited, except to the following claims, and their equivalents.

1. A biocompatible analyte sensing material for intradermal or subcutaneous application comprising:
a collection of microgel particles comprising one or more network crosslinker components, wherein the microgel particles are spherical in shape and have diameters within the range from about 30 micrometers to about 150 micrometers;
an endogenous or exogenous annealing agent that links the microgel particles together at points of physical contact in situ to form a covalently-stabilized scaffold of microgel particles having interstitial spaces therein; and
a plurality of analyte-specific fluorophores conjugated to the microgel particles, wherein the analyte-specific fluorophores, in the presence of the analyte and subject to excitation radiation, emit fluorescent light.

2. The biocompatible analyte sensing material of claim 1, wherein the analyte comprises glucose.

3. The biocompatible analyte sensing material of claim 1, wherein the analyte-specific fluorophores comprise at least one of Concanavalin A (Con A) or boronic acid conjugated fluorophores.

4. The biocompatible analyte sensing material of claim 1, wherein the analyte-specific fluorophores comprise glucose sensitive fluorophores based on diboronic or arylboronic acids.

5. The biocompatible analyte sensing material of claim 1, wherein the analyte-specific fluorophores comprise a glucose sensing aptamer linked to a fluorescent aptamer.

6. The biocompatible analyte sensing material of claim 1, wherein the covalently-stabilized scaffold of microgel particles is biodegradable or non-biodegradable.

7. (canceled)

8. The biocompatible analyte sensing material of claim 6, wherein the covalently-stabilized scaffold of microgel particles is non-biodegradable and photolytically degradable.

9. The biocompatible analyte sensing material of claim 1,
wherein the microgel particles containing the analyte-specific fluorophores are surrounded by a shell of hydrogel with no analyte-specific fluorophores.

10. A system for sensing analyte concentrations in live tissue of a subject comprising:
an analyte sensing material for intradermal or subcutaneous application comprising biocompatible microgel particles conjugated to analyte-specific fluorophores, wherein the analyte-specific fluorophores, in the presence of the analyte and when subject to excitation radiation, emit fluorescent light; and
an optical readout device configured to illuminate the analyte sensing material with excitation radiation and read the intensity of emitted fluorescent light.

11. The system of claim 10, wherein the optical readout device outputs or displays a concentration of the analyte based on the read intensity of emitted fluorescent light.

12. The system of claim 10, wherein a separate computing device receives data from the optical readout device and outputs or displays a concentration of the analyte based on the intensity of emitted fluorescent light.

13. The system of claim 10, wherein the optical readout device comprises a patch or bandage.

14. The system of claim 10, wherein the optical readout device comprises a wearable device.

15. (canceled)

16. The system of claim 10, further comprising a computer controlled pump configured to deliver a therapeutic to the subject based on the measured readings from the optical readout device.

17. The system of claim 10, wherein the analyte sensing material is biodegradable or non-biodegradable.

18. (canceled)

19. The biocompatible analyte sensing material of claim 17, wherein the covalently-stabilized scaffold of microgel particles is non-biodegradable and photolytically degradable.

20. A method of sensing an analyte in a subject comprising:
applying an analyte sensing material into an excision formed in the skin of a subject, the analyte sensing material comprising biocompatible microgel particles annealed or annealable to one another and conjugated to analyte-specific fluorophores, wherein the analyte-specific fluorophores, in the presence of the analyte and when subject to excitation radiation, emit fluorescent light;
exciting the analyte sensing material with excitation radiation and reading the emitted fluorescent light with an optical readout device.

21. The method of claim 20, wherein the optical readout device comprises a patch or bandage.

22. The method of claim 20, wherein the optical readout device comprises a wearable device.

23. (canceled)

24. The method of claim 20, wherein the optical readout device reads the intensity of the emitted fluorescent light.

25. The method of claim 20, wherein the optical readout device reads a pattern of the emitted fluorescent light.

26. A method of sensing an analyte in a subject comprising:
injecting an analyte sensing material into or under the skin of the subject, the analyte sensing material comprising biocompatible microgel particles annealed or annealable to one another and conjugated to analyte-specific fluorophores, wherein the analyte-specific fluorophores, in the presence of the analyte and when subject to excitation radiation, emit fluorescent light; and
exciting the analyte sensing material with excitation radiation and reading the emitted fluorescent light with an optical readout device.

27. The method of claim 26, wherein the optical readout device reads the intensity of the emitted fluorescent light.

28. The method of claim 26, wherein the optical readout device reads a pattern of the emitted fluorescent light.
